(19) United States
(12) Patent Application Publication
(10) Pub. No.: US 2023/0233223 A1
(43) Pub. Date: Jul. 27, 2023

(54) MEDICAL DEVICE AND SHUNT FORMATION METHOD

(71) Applicant: TERUMO KABUSHIKI KAISHA, Tokyo (JP)

(72) Inventors: Shinji KONDO, Hadano-shi (JP); Yusuke TAKAHASHI, Hadano-shi (JP); Tomoaki TAKEMURA, Kawasaki-shi (JP)

(73) Assignee: TERUMO KABUSHIKI KAISHA, Tokyo (JP)

(21) Appl. No.: 18/192,089

(22) Filed: Mar. 29, 2023

Related U.S. Application Data
(63) Continuation of application No. PCT/JP2021/035235, filed on Sep. 27, 2021.

(30) Foreign Application Priority Data
Sep. 30, 2020 (JP) 2020-164555

(51) Int. Cl. A61B 17/32

(52) U.S. Cl. CPC A61B 17/320068; A61B 2017/320078

(57) ABSTRACT

A medical device includes: an expansion body, a shaft portion, and an electrode portion, the expansion body includes a recessed portion defining a receiving space configured to receive a biological tissue, a proximal side upright portion of the recessed portion includes a first surface facing the receiving space and a second surface opposite to the first surface, a distal side upright portion of the recessed portion includes a third surface facing the receiving space and a fourth surface opposite to the third surface, the proximal side upright portion is an electrode arrangement portion in which the electrode portion is arranged, the distal side upright portion is an opposing surface portion opposing the electrode portion, the expansion body includes a heat insulation layer at least on the third surface or the fourth surface so as to oppose the electrode portion with the receiving space.

### CROSS-REFERENCES TO RELATED APPLICATIONS

This application is a continuation of International Application No. PCT/JP2021/035235 filed on Sep. 27, 2021, which claims priority to Japanese Patent Application No. 2020-164555 filed on Sep. 30, 2020, the entire content of both of which is incorporated herein by reference.

### TECHNOLOGICAL FIELD

The present disclosure generally relates to a medical device and a shunt forming method that impart energy to a biological tissue.

### BACKGROUND DISCUSSION

As a medical device, there is one in which an electrode portion is disposed on an expansion body that expands and contracts in a biological body, and treatment is performed by ablation, which is to cauterize a biological tissue by a high-frequency current from the electrode portion. As a treatment by ablation, the shunt treatment on the atrial septum is known. For the patients who suffer from heart failure, by forming, in an atrial septum, a shunt (puncture hole) serving as an escape route for increased atrial pressure, the shunt treatment enables heart failure symptoms to be alleviated. In the shunt treatment, the atrial septum is accessed using an intravenous approaching method, and the puncture hole is formed to a desired size.

In the medical device that performs treatment by ablation, a current flows from the electrode portion to the biological tissue, so that the temperature of the biological tissue or the vicinity of the electrode portion of the medical device becomes relatively high. As a result, steam pop generation or thrombus formation may cause a complication. The medical device disclosed in Japanese Patent Application Publication No. 2001-112772 A is a cautery probe configured by disposing a heating element in a cap, and is provided with a thermally insulative structure that reduces heat conduction from the heating element to a part of the outside surface of the cautery probe.

There is a medical device for performing ablation that has a heat generation site that is directly exposed outward such as the medical device used for the shunt treatment described above. At the time of cauterization, not only a heat generation site such as the electrode portion but also a biological tissue imparted with energy may have a relatively high temperature. For this reason, for the medical device in which the heat generation site is exposed outward, it is required to suppress heat from the heat generation site and the biological tissue heated by the heat generation site from propagating to the blood.

### SUMMARY

A medical device and a shunt forming method configured to suppress heat generated by cauterization from propagating to the blood are disclosed.

A medical device according to the present disclosure includes: an expansion body configured to expand and contract in a radial direction; an elongated shaft portion including a distal portion, the distal portion including a proximal end fixing portion to which a proximal end of the expansion body is fixed; and an electrode portion provided along the expansion body, in which the expansion body includes a recessed portion recessed radially inward when the expansion body expands and defining a receiving space configured to receive a biological tissue, the recessed portion includes a bottom portion positioned on a radial innermost side, a proximal side upright portion extending radially outward from a proximal end of the bottom portion, and a distal side upright portion extending radially outward from a distal end of the bottom portion, the proximal side upright portion includes a first surface facing the receiving space and a second surface opposite to the first surface, the distal side upright portion includes a third surface facing the receiving space and a fourth surface opposite to the third surface, one of the proximal side upright portion and the distal side upright portion is an electrode arrangement portion in which the electrode portion is arranged to face the receiving space, and an other of the proximal side upright portion and the distal side upright portion is an opposing surface portion opposing the electrode portion, and the expansion body includes a heat insulation layer at least on one or more of the first surface, the second surface, the third surface, and the fourth surface so as to oppose the electrode portion across the receiving space.

A medical device according to the present disclosure includes: an expansion body configured to expand and contract in a radial direction; an elongated shaft portion including a distal portion, the distal portion including a proximal end fixing portion to which a proximal end of the expansion body is fixed; an electrode portion provided along the expansion body; and a heat insulation cover portion covering at least a part of the expansion body, in which the expansion body includes a recessed portion recessed radially inward when the expansion body expands and defining a receiving space configured to receive a biological tissue, the recessed portion includes a bottom portion positioned on a radial innermost side, a proximal side upright portion extending radially outward from a proximal end of the bottom portion, and a distal side upright portion extending radially outward from a distal end of the bottom portion, the electrode portion is arranged in the recessed portion to face the receiving space, and the heat insulation cover portion is configured to cover at least a part of a surface of the recessed portion opposite to a surface facing the receiving space in a vicinity of the electrode portion.

A shunt forming method according to the present disclosure includes a method of forming a shunt in an atrial septum using a medical device including an expansion body configured to expand and contract in a radial direction, an elongated shaft portion including a distal portion, the distal portion including a proximal end fixing portion to which a proximal end of the expansion body is fixed, and an electrode portion provided along the expansion body, the method comprises: expanding the expansion body to include a recessed portion recessed radially inward and defining a receiving space configured to receive a biological tissue, arranging the recessed portion in a puncture hole formed in an atrial septum to receive the biological tissue surrounding the puncture hole in the receiving space defined by the recessed portion, and to bring the electrode portion into contact with the biological tissue, the electrode portion being arranged in the recessed portion to face the receiving space, and cauterizing the biological tissue by applying a voltage to the electrode portion in a state in which at least a part of the recessed portion includes a heat insulation layer or in a state in which at least a part of the recessed portion is covered with a heat insulation cover portion in a vicinity of the electrode portion.

In the medical device configured as described above, since the heat insulation layer is provided on the surface opposing the electrode portion across the receiving space, it is possible to make it difficult to propagate, to the blood, heat from the biological tissue raised to a high temperature by the energy imparted from the electrode portion or the heat generation site itself such as the electrode portion, and it is possible to reduce the risk of formation of a thrombus.

In the medical device configured as described above, since a part of the recessed portion on a surface opposite to a surface facing the receiving space is covered with the heat insulation cover portion at least in the vicinity of the electrode portion, it is possible to make it difficult to propagate, to the blood, heat from the biological tissue raised to a relatively high temperature by the energy imparted from the electrode portion, and it is possible to reduce the risk of formation of a thrombus.

In the shunt forming method configured as described above, when a voltage is applied to the electrode portion, since the recessed portion of the expansion body is insulated by the heat insulation layer or the heat insulation cover portion, it is possible to make it difficult to propagate, to the blood, heat associated with cauterization, and it is possible to reduce the risk of formation of a thrombus.

The expansion body may include a frame defining a shape of the expansion body, and the heat insulation layer disposed on a surface of the frame, which makes it possible to dispose the heat insulation layer while securing the flexibility of the expansion body.

The heat insulation layer may be disposed over the substantially entire surfaces of the inner surface in the expansion direction and the outer surface in the expansion direction of the frame, which makes it possible to enhance the heat insulation property of the expansion body, and to reliably reduce propagation of the heat associated with cauterization.

The heat insulation layer may be disposed on any two or more of the first surface, the second surface, the third surface, and the fourth surface to sandwich the receiving space, which makes it possible to reduce propagation of the heat associated with cauterization on both sides of the recessed portion.

The heat insulation layer may be disposed at the bottom portion on an inner surface in the expansion direction or an outer surface in the expansion direction, which makes it possible to reduce heat propagation at the bottom portion of the recessed portion.

The expansion body may include a tube covering the frame functioning as the heat insulation layer, which makes it possible to rather easily form the heat insulation layer simply by attaching the tube to the frame.

The expansion body may include a frame defining a shape of the expansion body, and the frame may include a heat insulation member including the heat insulation layer at least in a region of the recessed portion, which makes it possible to rather easily form the heat insulation layer by fixing the heat insulation member to the frame.

One of the proximal side upright portion and the distal side upright portion is an electrode arrangement portion in which the electrode portion is arranged to face the receiving space, and the other of the proximal side upright portion and the distal side upright portion is an opposing surface portion opposing the electrode portion, and the heat insulation cover portion may be disposed on the opposing surface portion on a surface opposite to a surface facing the receiving space, which makes it possible to help prevent the blood from coming into contact with the opposing surface portion, and therefore it is possible to reliably reduce propagation of the heat generated with cauterization.

The expansion body may include a frame defining a shape of the expansion body, the medical device may further include a second expansion body configured to expand and contract in a radial direction, including the heat insulation cover portion inside an expansion direction of the frame, and the heat insulation cover portion may cover at least a surface of the recessed portion of the frame opposite to a surface facing the receiving space. Due to this, the second expansion body also expands along with the expansion of the expansion body, and the recessed portion can be covered with the heat insulation cover portion on a surface opposite to the side facing the receiving space.

The second expansion body may include a second frame defining a shape of the second expansion body, and the heat insulation cover portion arranged on at least a part of the second frame, which makes it possible to dispose the heat insulation cover portion while securing the flexibility of the second expansion body.

The second expansion body may include a mesh in which a large number of wires are knitted, and the heat insulation cover portion arranged on at least a part of the mesh. Since the mesh is configured to flexibly deform in accordance with the shape of the expansion body, it is possible to enhance the heat insulation property by bringing the heat insulation cover portion into closer contact to the expansion body.

The second expansion body may include a balloon configured to expand in a radial direction, functioning as the heat insulation cover portion, which allows the inside of the expansion body to be covered with the balloon, and therefore it is possible to more reliably help prevent the surface of the recessed portion of the frame opposite to the surface facing the receiving space from coming into contact with the blood, and it is also possible to more reliably reduce the propagation of heat.

### BRIEF DESCRIPTION OF THE DRAWINGS

FIG. 1 is a front view showing an overall configuration of a medical device according to an embodiment.

FIG. 2 is an enlarged perspective view of the vicinity of an expansion body.

FIG. 3 is an enlarged front view of the vicinity of the expansion body.

FIG. 4 is a front view showing a state in which one of wire portions is extended flat.

FIG. 5 is a sectional view of the wire portion.

FIG. 6 is a view showing an expansion body stored in a storage sheath.

FIG. 7 is a view for schematically describing a state where the expansion body is disposed in an atrial septum, in which the medical device is shown in a front view and the biological tissue is shown in a sectional view, respectively.

FIG. 8 is an enlarged view of the vicinity of the expansion body in FIG. 7.

FIG. 9 is a view for schematically describing a state where a diameter of the expansion body is increased in the atrial septum from the state of FIG. 8.

FIG. 10 is an enlarged front view of the vicinity of an expansion body according to a first modification example.

FIG. 11 is an enlarged sectional view of the vicinity of a recessed portion of an expansion body according to a second modification example.

FIGS. 12A and 12B are an exploded view (FIG. 12A) and a front view (FIG. 12B) in which one of wire portions of an expansion body according to a third modification example is extended flat.

FIGS. 13A and 13B are enlarged sectional views of the vicinity of a recessed portion of an expansion body according to a fourth modification example.

FIG. 14 is an exploded view on a back side in which one of wire portions of an expansion body according to the fourth modification example is extended flat.

FIG. 15 is an enlarged sectional view of the vicinity of a recessed portion of an expansion body according to a fifth modification example.

FIGS. 16A and 16B are enlarged sectional views of the vicinity of an electrode portion of the expansion body according to the fifth modification example.

FIG. 17 is an enlarged sectional view of the vicinity of an electrode portion of an expansion body according to a sixth modification example.

FIGS. 18A and 18B are a front view (FIG. 18A) and a back view (FIG. 18B) in which one of wire portions of an expansion body according to a seventh modification example is extended flat.

FIG. 19 is an enlarged sectional view of the vicinity of a recessed portion of an expansion body according to the seventh modification example.

FIG. 20 is an enlarged view of the vicinity of an expansion body of a medical device according to the first modification example.

FIG. 21 is a front view in which a second frame of a second expansion body is extended flat.

FIG. 22 is an enlarged view of the vicinity of the expansion body in a case where an electrode portion of the medical device according to the first modification example is disposed at the bottom portion of the recessed portion.

FIG. 23 is a front view of a second expansion body disposed inside an expansion body in a medical device according to the second modification example.

FIG. 24 is an enlarged view of the vicinity of an expansion body of a medical device according to the second modification example.

FIG. 25 is a front view of a second expansion body disposed inside an expansion body in a medical device according to the third modification example.

FIGS. 26A and 26B are enlarged views of the vicinity of an expansion body of a medical device according to the third modification example.

FIG. 27 is an enlarged view of the vicinity of an expansion body including a second expansion body according to a modification example.

FIG. 28 is an enlarged view of the vicinity of an expansion body according to an eighth modification example.

FIG. 29 is an enlarged view of the vicinity of an expansion body according to a ninth modification example.

### DETAILED DESCRIPTION

Set forth below with reference to the accompanying drawings is a detailed description of embodiments of a medical device and a shunt forming method that impart energy to a biological tissue. In some cases, dimensional ratios in the drawings may be exaggerated and different from actual ratios for convenience of description. In addition, in the present specification, a side on which a medical device 10 is inserted into a biological lumen will be referred to as a “distal end” or a “distal side”, and an operating hand-side will be referred to as a “proximal end” or a “proximal side”.

The medical device 10 according to the embodiments described in this disclose may be configured as follows. A puncture hole Hh formed in an atrial septum HA of a patient's heart H is enlarged, and further, a maintenance treatment is performed so that the puncture hole Hh having an increased diameter is maintained to have an increased size.

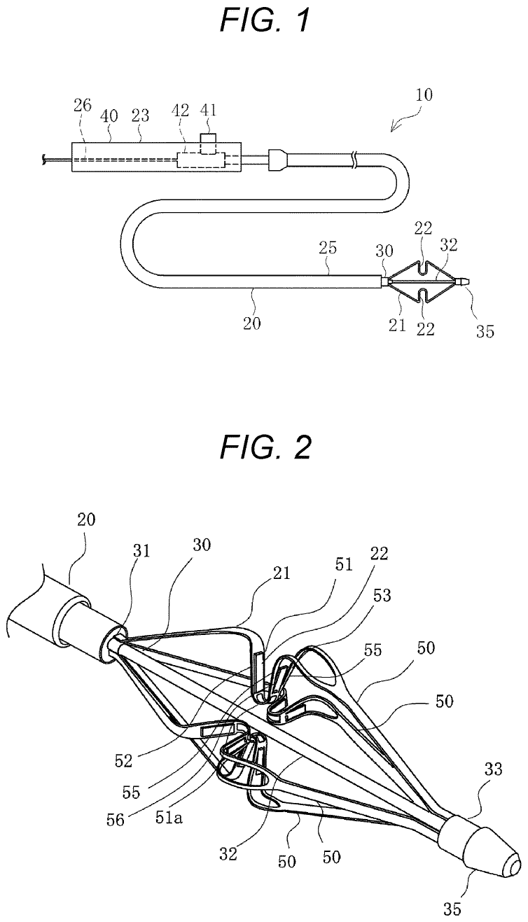

As shown in FIG. 1, the medical device 10 according to the present embodiment includes an elongated shaft portion 20, an expansion body 21 disposed in a distal portion of the shaft portion 20, and an operation unit 23 disposed in a proximal portion of the shaft portion 20. The expansion body 21 has an electrode portion 22, which is an energy transfer element for performing the above-described maintenance treatment.

The shaft portion 20 has a distal portion 30 including a proximal end fixing portion 31 to which the proximal end of the expansion body 21 is fixed and a distal end fixing portion 33 to which the distal end of the expansion body 21 is fixed. The distal portion 30 of the shaft portion 20 has a shaft extension portion 32 extending in the expansion body 21 from the proximal end fixing portion 31. The shaft portion 20 has a storage sheath 25 disposed at the outermost peripheral portion of the shaft portion 20. The expansion body 21 is movable forward and rearward from the storage sheath 25 in an axial direction. In a state where the storage sheath 25 is moved to the distal side of the shaft portion 20, the storage sheath 25 can internally store the expansion body 21. In a state where the expansion body 21 is stored, the storage sheath 25 is moved to the proximal side so that the expansion body 21 can be exposed.

The shaft portion 20 includes a pulling shaft 26. The pulling shaft 26 is disposed from the proximal end of the shaft portion 20 to the shaft extension portion 32, and the distal portion is fixed to a distal member 35. A proximal portion of the pulling shaft 26 is drawn out (i.e., extends) to the proximal side of the operation unit 23.

The distal portion of the pulling shaft 26 is fixed to the distal member 35. The distal member 35 may not be fixed to the expansion body 21. In this manner, by the distal member not being fixed to the expansion body 21, the distal member 35 can pull the expansion body 21 in a contracting direction. In addition, when the expansion body 21 is stored in the storage sheath 25, the distal member 35 can be separated to the distal side from the expansion body 21. Accordingly, the expansion body 21 can be rather easily moved in an axial direction, and storage capability can be improved.

The operation unit 23 has a housing 40 configured to be held by an operator, an operation dial 41 that can be rotationally operated by the operator, and a conversion mechanism 42 operated in conjunction with the rotation of the operation dial 41. The pulling shaft 26 is held inside the operation unit 23 by the conversion mechanism 42. In conjunction with the rotation of the operation dial 41, the conversion mechanism 42 can move the held pulling shaft 26 forward and backward along the axial direction. For example, a rack and pinion mechanism can be used as the conversion mechanism 42.

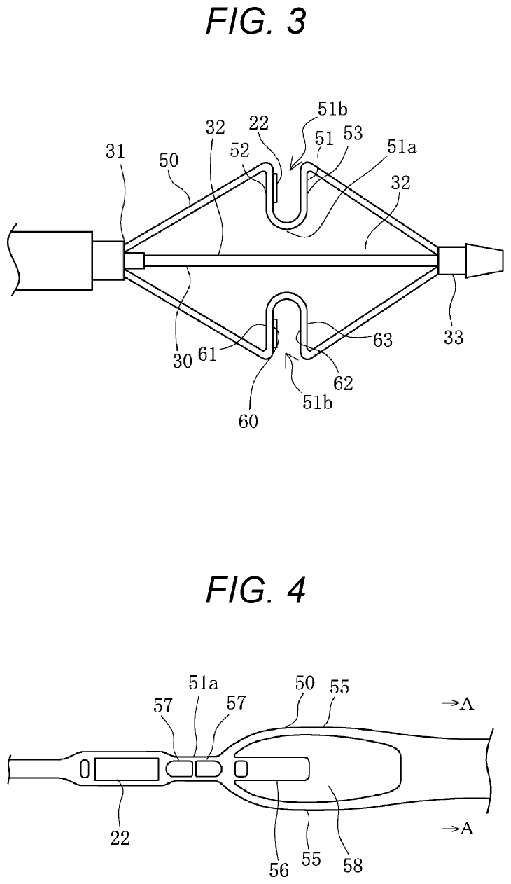

The expansion body 21 will be described in more detail. As shown in FIGS. 2 and 3, the expansion body 21 has a plurality of wire portions 50 in a circumferential direction. In the present embodiment, for example, four of the wire portions 50 are disposed in the circumferential direction. The wire portions 50 are respectively configured to expand and contract in a radial direction. A proximal portion of the wire portion 50 extends to a distal side from the proximal end fixing portion 31. A distal portion of the wire portion 50 extends from a proximal portion to a proximal side of the distal member 35. The wire portion 50 can be inclined to increase in the radial direction from both end portions toward a central portion in an axial direction. In addition, in the wire portion 50, the central portion in the axial direction has a recessed portion 51 recessed radially inward of the expansion body 21. A radially innermost portion of the recessed portion 51 is a bottom portion 51a. The recessed portion 51 defines a receiving space 51b configured to receive a biological tissue when the expansion body 21 expands.

The recessed portion 51 includes a proximal side upright portion 52 extending radially outward from the proximal end of the bottom portion 51a and a distal side upright portion 53 extending radially outward from the distal end of the bottom portion 51a. In the distal side upright portion 53, a central portion in a width direction has a slit shape. The distal side upright portion 53 has an outer edge portion 55 on both sides and a backrest portion 56 of the central portion.

As shown in FIG. 4, the wire portion 50 has a through-hole 57 on both sides in the extending direction of the portion to be the bottom portion 51a. A space portion 58 is formed between the outer edge portions 55 on both sides, and the backrest portion 56 is provided so as to protrude into the space portion 58.

In the recessed portion 51, the proximal side upright portion 52 to be disposed has a first surface 60 facing the receiving space 51b and a second surface 61 opposite to the first surface 60. The proximal side upright portion 52 is an electrode arrangement portion in which the electrode portion 22 is arranged. The distal side upright portion 53 of the recessed portion 51 is an opposing surface portion opposing the electrode portion 22, and has a third surface 62 facing the receiving space 51b and a fourth surface 63 opposite to the third surface 62.

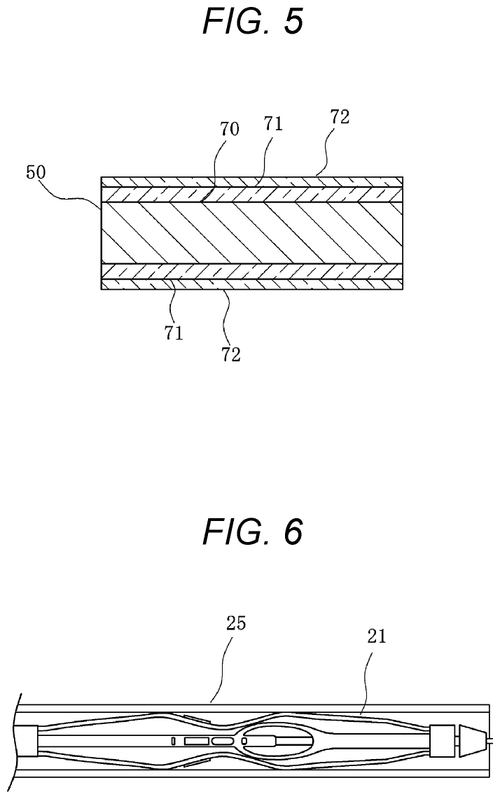

As shown in FIG. 5, the wire portion 50 includes a heat insulation layer 71 on a surface of a frame 70 made of metal and defining a shape of the expansion body 21, and further includes a biocompatible coating 72 on a surface of the heat insulation layer 71. The heat insulation layer 71 can be disposed on any of both surfaces of the opposing surface portion opposing the electrode arrangement portion where at least the electrode portion 22 is arranged. In the present embodiment, since the heat insulation layer 71 and the biocompatible coating 72 are disposed over substantially the entire surfaces of the inner surface and the outer surface of the frame 70 in the expansion direction, the first surface 60 and the second surface 61 of the electrode arrangement portion and the third surface 62 and the fourth surface 63 of the opposing surface portion each include the heat insulation layer 71.

The frame 70 can be, for example, made or fabricated from a metal material. For example, the metal material of the frame 70 can be a titanium-based (Ti—Ni, Ti—Pd, or Ti—Nb—Sn) alloy, a copper-based alloy, stainless steel, β-titanium steel, or a Co—Cr alloy. An alloy having a spring property such as a nickel titanium alloy may also be used as the material. However, a material of the frame 70 is not limited, and the frame 70 may be formed of other materials.

The heat insulation layer 71 can be, for example, made or fabricated of a resin or foamed plastic having a relatively low thermal conductivity. For example, the resin or the foamed plastic can be polyether ether ketone (PEEK), polyimide, PEBAX, an epoxy resin, a polytetrafluoroethylene resin (PTFE), or polyurethane. The biocompatible coating 72 can be, for example, made or fabricated of polymethoxyethyl acrylate (PMEA) or the like. The heat insulation layer 71 and the biocompatible coating 72 may be made or fabricated of other materials.

For example, the wire portion 50 forming the expansion body 21 has a flat plate shape cut out from a cylinder. The wire forming the expansion body 21 can have, for example, a thickness of 50 μm to 500 μm and a width of 0.3 mm to 2.0 mm. However, the wire may have a dimension outside this range. In addition, the wire portion 50 may have a circular shape in a cross section, or may have other shapes in a cross section.

The electrode portion 22 is disposed along the proximal side upright portion 52. Accordingly, when the recessed portion 51 is disposed in the atrial septum HA, the energy from the electrode portion 22 may be transferred to the atrial septum HA from the right atrium side.

For example, the electrode portion 22 is configured to include a bipolar electrode that receives electric energy from an energy supply device serving as an external device. In this case, electricity is supplied to the electrode portion 22 disposed in each of the wire portions 50. The electrode portion 22 and the energy supply device are connected to each other by a conducting wire coated with an insulating coating material. The conducting wire is drawn outward (i.e., extends) via the shaft portion 20 and the operation unit 23, and is connected to the energy supply device.

Alternatively, the electrode portion 22 may be configured to serve as a monopolar electrode. In this case, the electricity is supplied from a counter electrode plate prepared outside a body. In addition, the electrode portion 22 may alternatively be a heating element (electrode chip) that generates heat by receiving high-frequency electric energy from the energy supply device. In this case, the electricity is supplied to the heating element disposed in each of the wire portions 50. Furthermore, the electrode portion 22 can be configured to include an energy transfer element that applies energy to the puncture hole Hh, such as, for example, a heater including an electric wire which provides heating and cooling operation or generating frictional heat by using microwave energy, ultrasound energy, coherent light such as laser, a heated fluid, a cooled fluid, or a chemical medium. A specific form of the energy transfer element is not particularly limited.

It is preferable that the shaft portion 20 is made or fabricated, for example, of a material having a certain degree of flexibility. For example, the materials of the shaft portion 20 may include polyolefin such as polyethylene, polypropylene, polybutene, ethylene-propylene copolymer, ethylene-vinyl acetate copolymer, ionomer, and a mixture of the above-described two or more materials, fluororesin such as soft polyvinyl chloride resin, polyamide, polyamide elastomer, polyester, polyester elastomer, polyurethane, and polytetrafluoroethylene, polyimide, PEEK, silicone rubber, or latex rubber.

For example, the pulling shaft 26 can be, made or fabricated of the materials in which an elongated wire formed of a super elastic alloy such as a nickel-titanium alloy and a copper-zinc alloy, a metal material such as stainless steel, or a resin material having relatively high rigidity coated with a resin material such as polyvinyl chloride, polyethylene, polypropylene, and ethylene-propylene copolymer.

For example, the distal member 35 can be, for example, made or fabricated of a polymer material such as polyolefin, polyvinyl chloride, polyamide, polyamide elastomer, polyurethane, polyurethane elastomer, polyimide, and fluororesin or a mixture of polymer materials. Alternatively, the distal member 35 can be made or fabricated of a multilayer tube (or tubular member) containing two or more polymer materials.

As shown in FIG. 6, the expansion body 21 housed in the storage sheath 25 is in a state of contracting in the radial direction. When the expansion body 21 moves in the axial direction with respect to the storage sheath 25 and is exposed outward of the storage sheath 25, the expansion body 21 is in an expanded state as shown in FIG. 3.

In the present embodiment, four wire portions 50 are disposed in the circumferential direction, and four electrode portions 22 are also disposed, but more than four wire portions 50 having the recessed portion 51 and more than four electrode portions 22 may be disposed. The same applies to the modification examples described below.

In the present embodiment, the electrode portion 22 is disposed in the proximal side upright portion 52, but some or all of the electrode portions 22 may be disposed in the distal side upright portion 53. In this case, the distal side upright portion 53 serves as the electrode arrangement portion, the proximal side upright portion 52 serves as the opposing surface portion, and the heat insulation layer 71 is disposed at least on the first surface 60 or the second surface 61 opposing the electrode portion 22 across the receiving space 51b. The same applies to the modification examples described below in which the heat insulation layer is disposed.

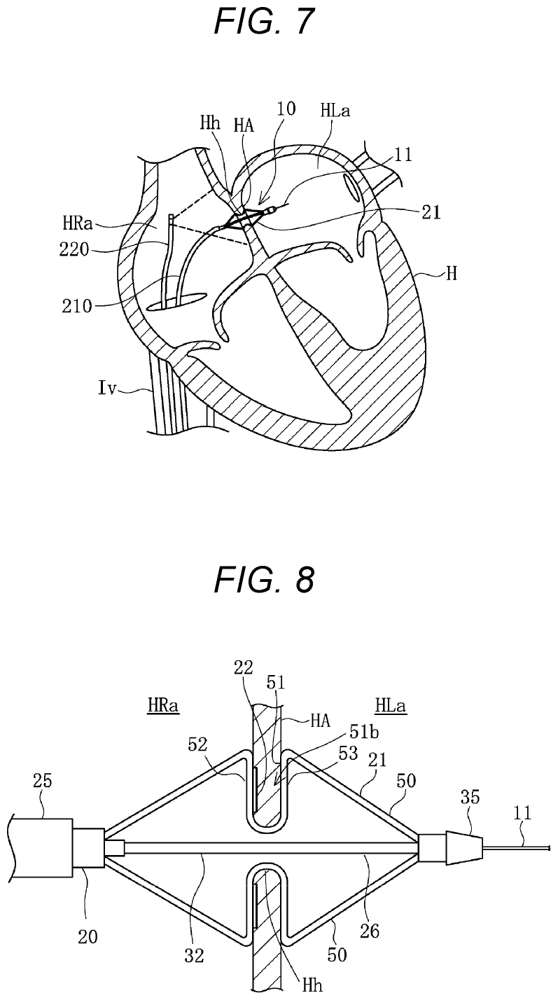

A treatment method using the medical device 10 will be described. The treatment method according to the present embodiment is performed on a patient suffering from a heart failure (left heart failure). More specifically, as shown in FIG. 7, the treatment method is performed on the patient suffering from a chronic heart failure, who has relatively high blood pressure in a left atrium Hla due to myocardial hypertrophy appearing in a left ventricle of the heart H and increased stiffness (hardness) of the heart muscle.

The treatment method according to the present embodiment includes forming the puncture hole Hh in the atrial septum HA (S1), disposing the expansion body 21 in the puncture hole Hh (S2), enlarging the diameter of the puncture hole Hh by using the expansion body 21 (S3), confirming hemodynamics in the vicinity of the puncture hole Hh (S4), performing the maintenance treatment for maintaining the size of the puncture hole Hh (S5), and confirming the hemodynamics in the vicinity of the puncture hole Hh after the maintenance treatment is performed (S6).

When the puncture hole Hh is formed, an operator delivers an introducer 210 in which a guiding sheath and a dilator are combined with each other, to the vicinity of the atrial septum HA. For example, the introducer 210 can be delivered to a right atrium Hra via an inferior vena cava Iv. In addition, the introducer can be delivered using the guide wire 11. The operator can insert the guide wire 11 into the dilator, and can deliver the introducer along the guide wire 11. The introducer and the guide wire 11 can be inserted into a living body by using a method such as using a blood vessel introducer.

In the forming of the puncture hole Hh in the atrial septum HA (S1), the operator causes a puncture device to penetrate from the right atrium Hra side toward the left atrium Hla side, thereby forming the puncture hole Hh. For example, a device such as a wire having a sharp distal end can be used as the puncture device. The puncture device is inserted into the dilator, and is delivered to the atrial septum HA. The puncture device can be delivered to the atrial septum HA instead of the guide wire 11 after the guide wire 11 is removed from the dilator.

In the disposing of the expansion body 21 in the puncture hole Hh (S2), the medical device 10 is first delivered to the vicinity of the atrial septum HA along the guide wire 11 inserted in advance. At this time, the distal portion of the medical device 10 penetrates the atrial septum HA, and reaches the left atrium Hla. In addition, when the medical device 10 is inserted, the expansion body 21 is in a state of being stored in the storage sheath 25.

Next, as shown in FIG. 8, the storage sheath 25 is moved to the proximal side so that the expansion body 21 is exposed. In this manner, the diameter of the expansion body 21 increases, and the recessed portion 51 is arranged in the puncture hole Hh of the atrial septum HA and receives the biological tissue surrounding the puncture hole Hh in the receiving space 51b.

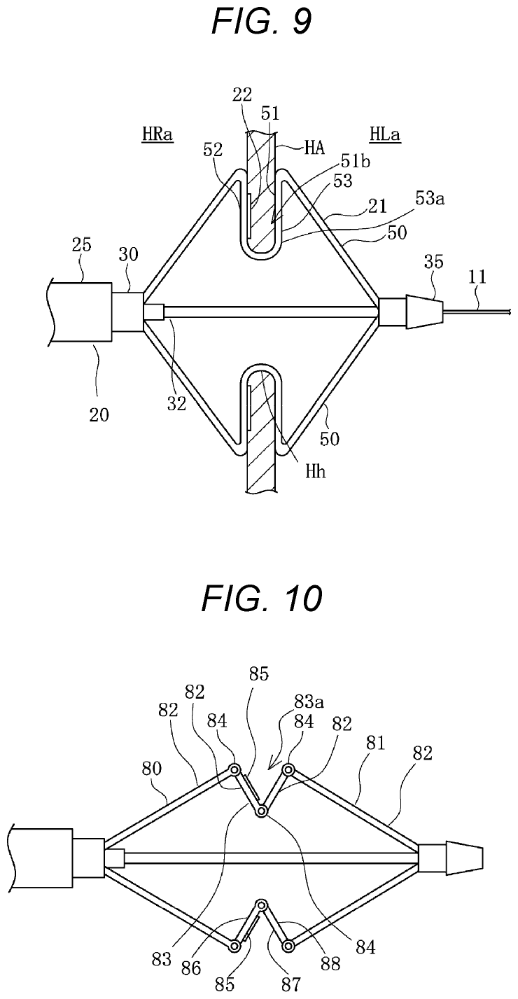

In the enlarging of the diameter of the puncture hole Hh by using the expansion body 21 (S3), the operator operates the operation unit 23 in a state where the receiving space 51b receives the biological tissue, and the pulling shaft 26 is moved to the proximal side. In this manner, as shown in FIG. 9, the expansion body 21 further expands in the radial direction, and the puncture hole Hh is widened in the radial direction.

After the puncture hole Hh is enlarged, the hemodynamics is confirmed in the vicinity of the puncture hole Hh (S4). As shown in FIG. 7, the operator delivers a hemodynamics confirming device 220 to the right atrium Hra by way of the inferior vena cava Iv. For example, an echo catheter can be used as the hemodynamics confirming device 220. The operator can display an echo image acquired by the hemodynamics confirming device 220 on a display apparatus such as a display, and can confirm a blood volume passing through the puncture hole Hh, based on a result of the echo image.

Next, the operator performs the maintenance treatment for maintaining the size of the puncture hole Hh (S5). In the maintenance treatment, high-frequency energy is imparted to an edge portion of the puncture hole Hh through the electrode portion 22, thereby cauterizing (heating and cauterizing) the edge portion of the puncture hole Hh by using the high-frequency energy.

At the time of cauterization, heat is generated at the edge portion of the puncture hole Hh by the high-frequency energy from the electrode portion 22, but since the expansion body 21 has the heat insulation layer 71, it is possible to suppress the propagation of the heat generated by cauterization to the blood, which makes it possible to suppress formation of a thrombus due to cauterization.

When the biological tissue in the vicinity of the edge portion of the puncture hole Hh is cauterized through the electrode portion 22, a degenerated portion having the degenerated biological tissue is formed in the vicinity of the edge portion. The biological tissue in the degenerated portion is in a state where elasticity is lost. Accordingly, the puncture hole Hh can maintain a shape widened by the expansion body 21.

After the maintenance treatment is performed, the hemodynamics are confirmed again in the vicinity of the puncture hole Hh (S6). In a case where the blood volume passing through the puncture hole Hh reaches a desired volume, the operator decreases the diameter of the expansion body 21. After the expansion body 21 is stored in the storage sheath 25, the expansion body 21 is removed from the puncture hole Hh. Furthermore, the whole medical device 10 is removed outward of the living body, and the treatment is completed.

Next, a modification example of the expansion body will be described. As shown in FIG. 10, an expansion body 80 of the first modification example includes a frame 81 defining a shape of the expansion body 80. The frame 81 is formed by linking a plurality of heat insulation members 82 by a hinge portion 84. The heat insulation member 82 can be formed of fine ceramics, zirconia, or the like having a relatively low thermal conductivity. However, the heat insulation member 82 may be formed of a material other than these materials.

The expansion body 80 has a recessed portion 83 that deforms with expansion and contraction, and an electrode portion 85 is disposed in the recessed portion 83. The recessed portion 83 is configured to receive the edge portion of the puncture hole Hh in a receiving space 83a. The heat insulation member 82 is formed of a material having a relatively low flexibility, and therefore, by disposing the hinge portion 84, it is possible to expand and contract the expansion body 80 without damaging the biological tissue. Since the entire expansion body 80 is formed of the heat insulation member 82, each of a first surface 86, a second surface 87, a third surface 88, and a fourth surface 89 has a heat insulation layer. By forming the entire expansion body 80 with the heat insulation member 82, it is possible to suppress propagation of heat generated by cauterization to blood when high-frequency energy is output from the electrode portion 85 in a state where the biological tissue is received in the receiving space 83a of the recessed portion 83.

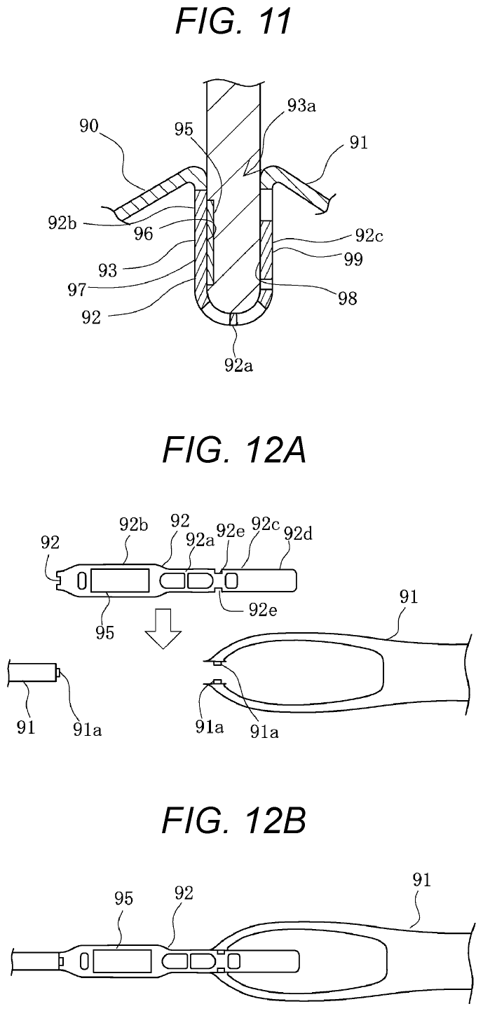

As shown in FIG. 11, an expansion body 90 of the second modification example includes a frame 91 defining a shape of the expansion body 90. The frame 91 has a heat insulation member 92 in a portion of a recessed portion 93. The heat insulation member 92 can be formed of fine ceramics, zirconia, or the like having low thermal conductivity. However, the heat insulation member 92 may be formed of a material other than these materials. Since the recessed portion 93 is formed of the heat insulation member 92, each of a first surface 96, a second surface 97, a third surface 98, and a fourth surface 99 has a heat insulation layer.

As shown in FIGS. 12A and 12B, the heat insulation member 92 has portions to be a bottom portion 92a, a proximal side upright portion 92b, a distal side upright portion 92c, and a backrest portion 92d, and the proximal side upright portion 92b has an electrode portion 95. The heat insulation member 92 includes a plurality of engaging portions 92e. The frame 91 includes an engaged portion 91a to which the engaging portion 92e of the heat insulation member 92 is engaged. By engaging the engaging portion 92e to the engaged portion 91a, it is possible to integrate the heat insulation member 92 with the frame 91, which makes it possible to reduce the thermal conductivity of the portion of the recessed portion 93 in the expansion body 90 and to suppress propagation of heat generated by cauterization to blood when high-frequency energy is output from the electrode portion 95 in a state where the biological tissue is received in the receiving space 93a of the recessed portion 93.

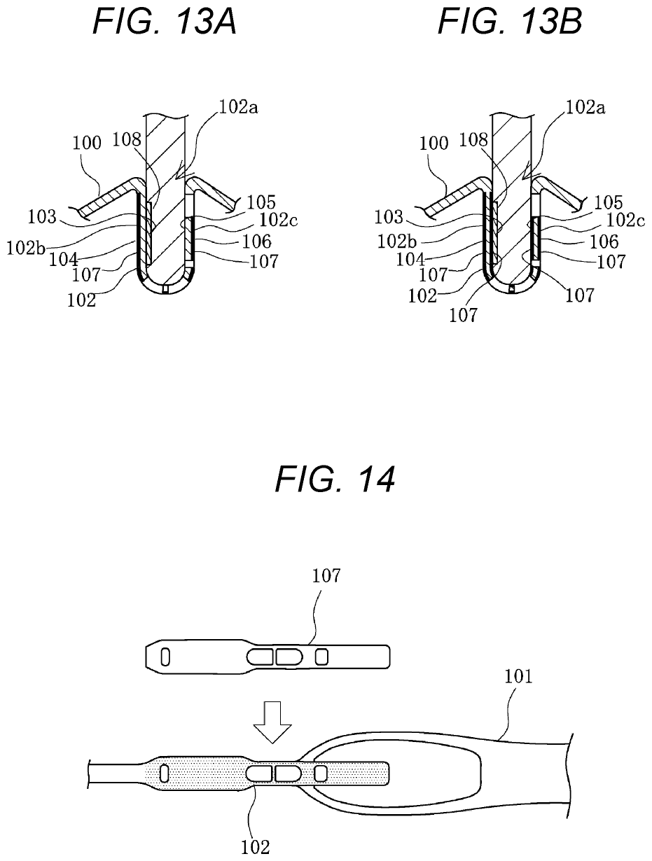

A heat insulation layer 107 may be disposed on two surfaces sandwiching a receiving space 102a among a first surface 103, a second surface 104, a third surface 105, and a fourth surface 106. As shown in FIG. 13A, in an expansion body 100 of the third modification example, the heat insulation layer 107 is disposed on a surface of a recessed portion 102 opposite to a side facing the receiving space 102a. That is, the heat insulation layer 107 is disposed on the second surface 104 and the fourth surface 106 so as to sandwich the receiving space 102a. The two surfaces (i.e., two surfaces selected from the first surface 103, the second surface 104, the third surface 105, and the fourth surface 106) having the heat insulation layer 107 may be a combination other than this as long as the receiving space 102a is sandwiched between the two surfaces having the heat insulation layer 107, and for example, may be disposed on the first surface 103 and the fourth surface 106.

As shown in FIG. 13B, the heat insulation layer 107 may be provided on the first surface 103 and the third surface 105 in addition to the second surface 104 and the fourth surface 106. In any case, since the propagation of heat in the recessed portion 102 can be reduced by the heat insulation layer 107, it is possible to suppress propagation of heat generated by cauterization to blood when high-frequency energy is output from the electrode portion 108 in a state where the biological tissue is received in the receiving space 102a of the recessed portion 102.

As shown in FIG. 14, the heat insulation layer 107 is formed as a sheet having a shape of a portion of the recessed portion 102. The heat insulation layer 107 is bonded and fixed to a frame 101 in a portion of the recessed portion 102 hatched in the figure. The fixing of the heat insulation layer 107 is not limited to bonding, and may be fixed to the frame 101 using a wire or the like.

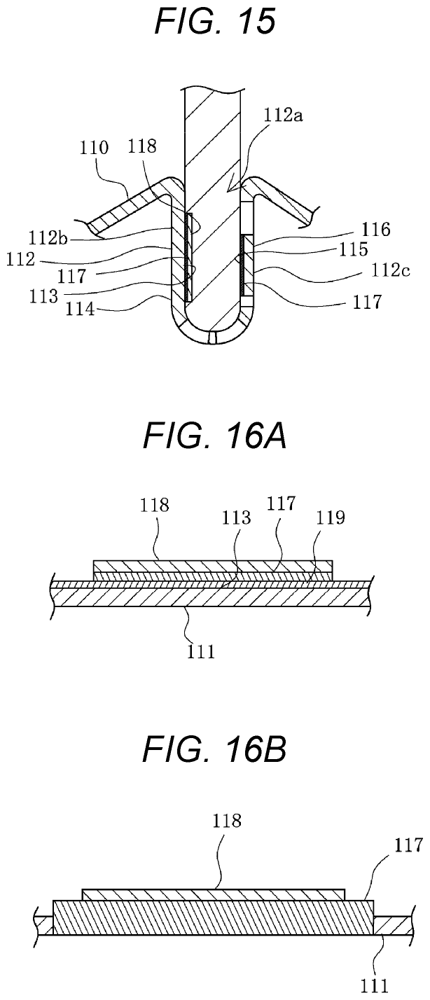

As shown in FIG. 15, an expansion body 110 of the fourth modification example includes an electrode portion 118 in a proximal side upright portion 112b of a recessed portion 112, and a distal side upright portion 112c is an opposing surface portion. A heat insulation layer 117 is disposed on a first surface 113 on a contact surface with the electrode portion 118 and a third surface 115.

As shown in FIG. 16A, the heat insulation layer 117 on the first surface 113 is configured to be disposed between a flexible substrate 119 disposed along the surface of a frame 111 defining the shape of the expansion body 110 and the electrode portion 118. As shown in FIG. 16B, the heat insulation layer 117 having the electrode portion 118 fixed to the surface thereof may be fixed to the frame 111. In this case, the heat insulation layer 117 is fixed to the frame 111 by bonding, wire, or the like.

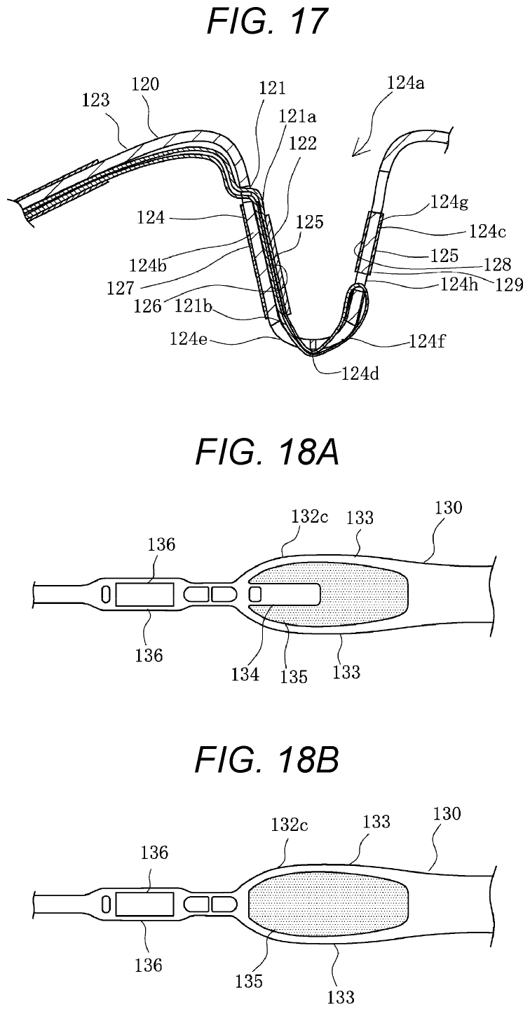

As shown in FIG. 17, an expansion body 120 of the fifth modification example includes, as a separate body from a frame 123, an electrode assembly 121 including an electrode portion 122. The frame 123 includes a recessed portion 124 defining a receiving space 124a, and the recessed portion 124 includes a proximal side upright portion 124b and a distal side upright portion 124c. A proximal side through-hole 124e and a distal side through-hole 124f are formed in a bottom portion 124d of the recessed portion 124. A backrest portion through-hole 124h is formed in a backrest portion 124g.

The electrode assembly 121 includes an inner wiring portion 121a that is exposed to the receiving space 124a and in which the electrode portion 122 is arranged. The electrode assembly 121 includes a folded-back wiring portion 121b that passes through the proximal side through-hole 124e and the distal side through-hole 124f and is folded back at the backrest portion through-hole 124h on the side distal of the inner wiring portion 121a. The folded-back wiring portion 121b passes through the proximal side through-hole 124e and is disposed between the inner wiring portion 121a and the proximal side upright portion 124b.

The expansion body 120 includes a tube (tubular member) 125 covering and fixing the proximal side upright portion 124b and the folded-back wiring portion 121b. The tube 125 is formed of a material such as nylon elastomer that contracts by heat. The tube 125 has a relatively low thermal conductivity and functions as a heat insulation layer. The tube 125 is also disposed in the backrest portion 124g. Due to this, a first surface 126, a second surface 127, a third surface 128, and a fourth surface 129 of a recessed portion 124 are covered with the tube 125, which is a heat insulation layer, and it is possible to suppress the heat generated by cauterization from propagating to the blood.

Next, the fifth modification example of the expansion body will be described. As shown in FIGS. 18A and 18B, an expansion body 130 of the fifth modification example has a heat insulation cover portion 135 disposed in a region surrounded by outer edge portions 133 on both sides of a distal side upright portion 132c and on the back surface side of a backrest portion 134. A frame 131 of the expansion body 130 is formed of a metal material. The heat insulation cover portion 135 is formed of a material having flexibility and a relatively low thermal conductivity. Such material for the heat insulation cover portion 135 can be, for example, rubber or foamed rubber such as silicone rubber. However, the material of the heat insulation cover portion 135 may be other than this.

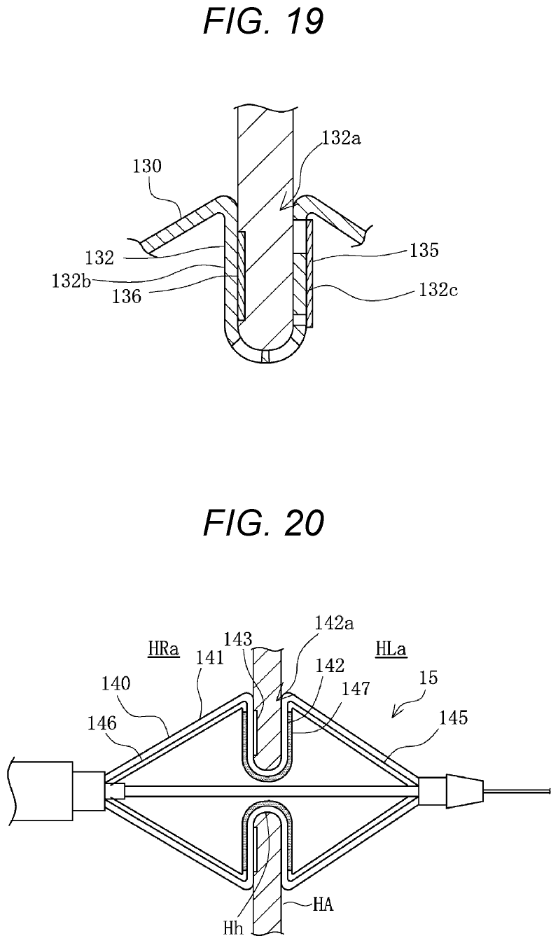

As shown in FIG. 19, an electrode portion 136 is disposed in a proximal side upright portion 132b of a recessed portion 132, and a surface opposite to a surface facing a receiving space 132a in the distal side upright portion 132c to be an opposing surface portion is covered with the heat insulation cover portion 135.

When high-frequency energy is output from the electrode portion 136 to the biological tissue, the edge portion of the puncture hole Hh is raised to a relatively high temperature, and the heat propagates to the opposing surface portion. The heat insulation cover portion 135 is disposed on the opposing surface portion, and a contact surface with the blood is covered, which suppresses the heat generated by cauterization from propagating to the blood by the heat insulation cover portion 135. Also in the present example, the electrode portion 136 may be disposed in the distal side upright portion 132c. The same applies to the modification example below in which the heat insulation cover portion is disposed.

Next, a modification example of the medical device will be described. As shown in FIG. 20, a medical device 15 of the first modification example includes a second expansion body 145 inside the expansion direction of a frame 141 included in an expansion body 140. The second expansion body 145 includes a second frame 146 along the inside of the frame 141 in the expansion direction, and a heat insulation cover portion 147. An electrode portion 143 is disposed so as to face a receiving space 142a of a recessed portion 142.

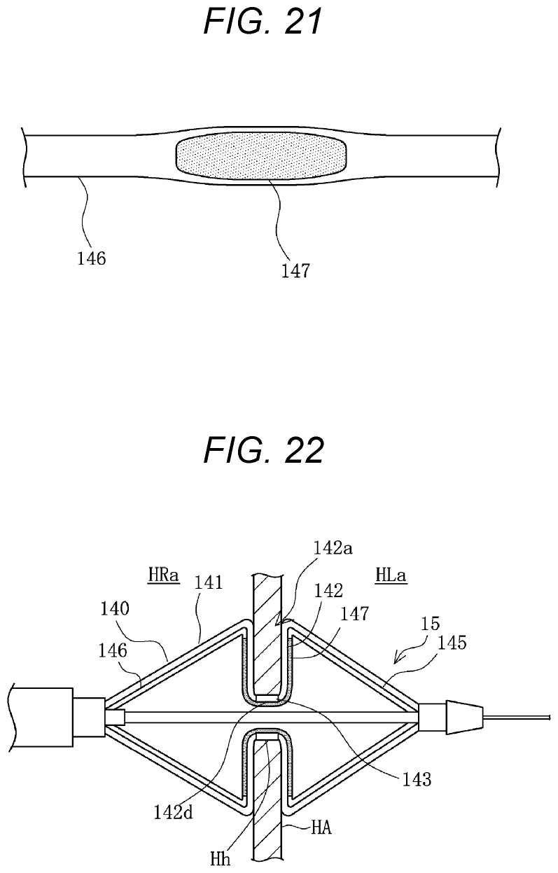

As shown in FIG. 21, the second frame 146 of the second expansion body 145 has a shape along the frame 141, and the heat insulation cover portion 147 is disposed in a portion covering the recessed portion 142. The second frame 146 is configured to expand and contract in a radial direction together with the frame 141. When the frame 141 expands in the radial direction, the second frame 146 also expands in the radial direction, and the heat insulation cover portion 147 can cover the recessed portion 142 of the frame 141 in close contact with a surface opposite to the surface facing the receiving space 142a. In this manner, the heat insulation cover portion 147 may be disposed on the second expansion body 145 separate from the frame 141 to cover the recessed portion 142 on the surface opposite to the surface facing the receiving space 142a.

In the medical device 15 of the first modification example, as shown in FIG. 22, the electrode portion 143 may be disposed at a bottom portion 142d of the recessed portion 142. Also in this case, the heat insulation cover portion 147 included in the second frame 146 can cover the recessed portion 142 of the frame 141 in relatively close contact with a surface opposite to the surface facing the receiving space 142a.

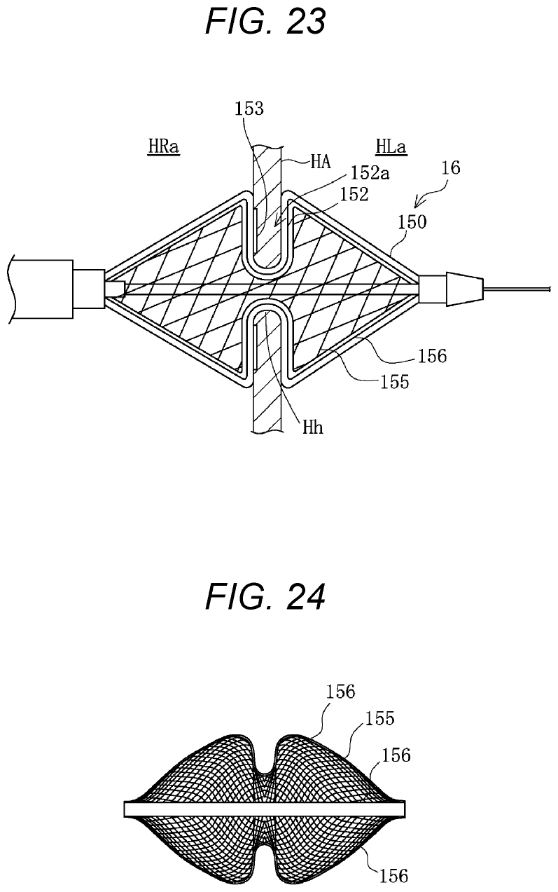

As shown in FIG. 23, a medical device 16 of the second modification example includes a second expansion body 155 including a mesh in which a large number of wires are knitted inside the expansion direction of an expansion body 150. An electrode portion 153 is disposed to face a receiving space 152a of a recessed portion 152. The second expansion body 155 is configured to expand and contract in a radial direction together with the expansion body 150. As shown in FIG. 24, the second expansion body 155 has an outer shape along the inside of the expansion body 150 in a state of expanding in the radial direction, and includes a heat insulation cover portions 156 at four locations corresponding to circumferential positions of a frame 151. When the second expansion body 155 expands together with the expansion body 150, the heat insulation cover portion 156 can cover the recessed portion 152 of the frame 151 in relatively close contact with a surface opposite to the surface facing the receiving space 152a. In this manner, the second expansion body 155 including the heat insulation cover portion 156 may be provided inside the expansion body 150 to cover the recessed portion 152 on the surface opposite to the surface facing the receiving space 152a.

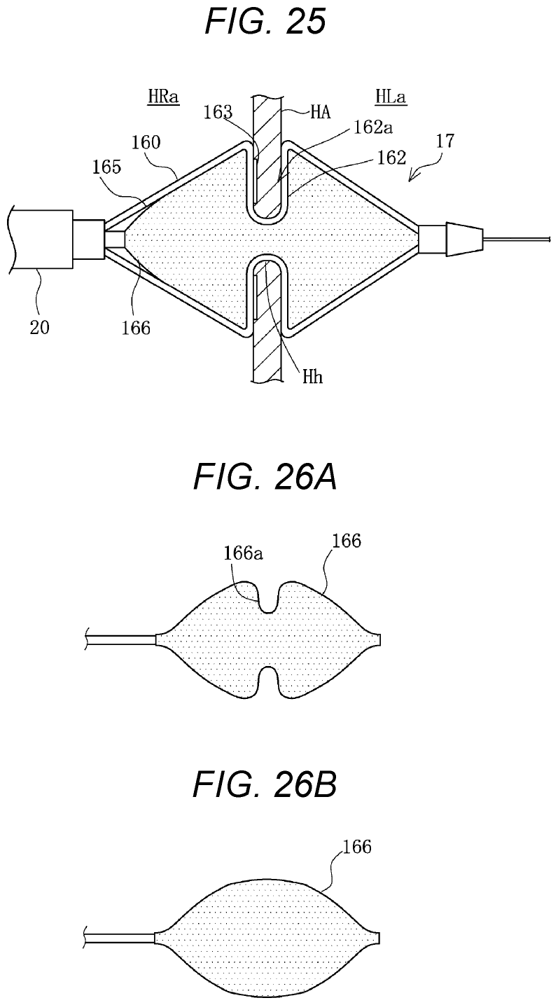

As shown in FIG. 25, in a medical device 17 of the third modification example has a balloon 166 disposed as a second expansion body 165 functioning as a heat insulation cover portion inside an expansion direction of an expansion body 160. An electrode portion 163 is disposed to face a receiving space 162a of a recessed portion 162. The balloon 166 is configured to be expanded in a radial direction by injecting an expansion fluid through an expansion lumen disposed in the shaft portion 20. As shown in FIG. 26A, the balloon 166 has an outer shape having a recessed portion 166a along the inside of the expansion body 160. The balloon 166 may have a shape not including a recessed portion as shown in FIG. 26B as long as the balloon 166 can flexibly deform in accordance with the shape of the expansion body 160.

By expanding the balloon 166 in a state where the expansion body 160 expands and bringing a surface of the balloon 166 into relatively close contact with the inside of the expansion body 160, it is possible to help prevent the recessed portion 162 of the expansion body 160 from coming into contact with blood, which makes it possible to suppress the heat generated when cauterizing the biological tissue by the electrode portion 163 from propagating to the blood, and to suppress formation of a thrombus.

As shown in FIG. 27, a balloon 167 only needs to cover the portion of the recessed portion 162 of the expansion body 160 from inside, and needs not have a size that extends or covers the entire expansion body 160.

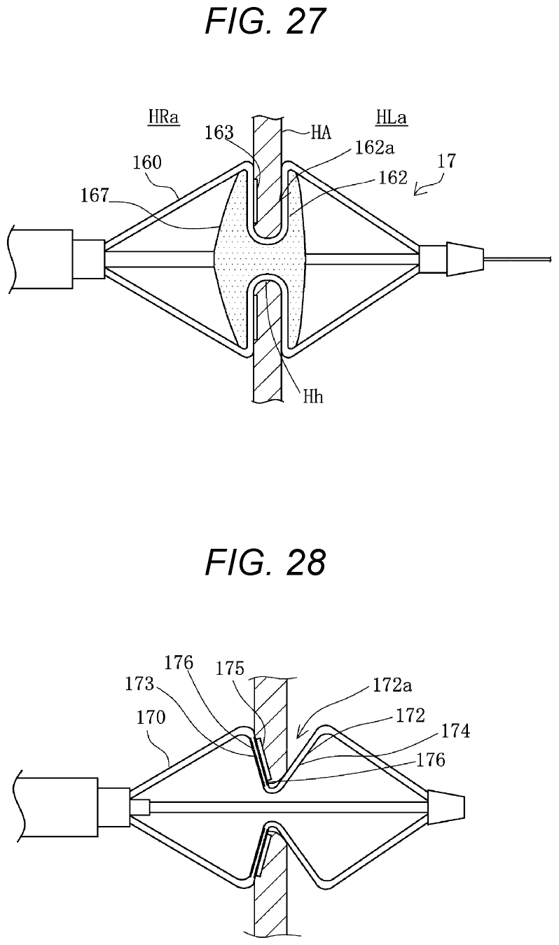

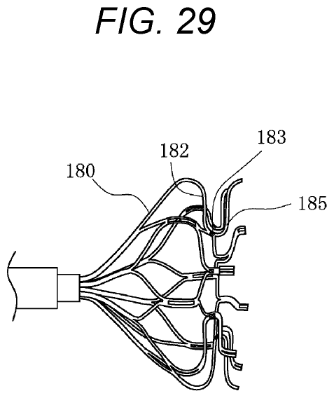

The expansion body is not limited to one that holds a biological tissue. An expansion body 170 shown in FIG. 28 receives a biological tissue in a receiving space 172a of a recessed portion 172, but does not hold the biological tissue. The recessed portion 172 includes a proximal side upright portion 173 and a distal side upright portion 174. In this state, high-frequency energy is imparted to the biological tissue from an electrode portion 175. The proximal side upright portion 173 of the recessed portion 172 is provided with a heat insulation layer 176, and it is possible to suppress heat generated by cauterization from propagating to blood.

The expansion body is not limited to one formed of a plurality of wire portions. An expansion body 180 shown in FIG. 29 is formed in a mesh shape in which wires are branched and merged. The expansion body 180 includes a recessed portion 182, and an electrode portion 183 is disposed. The portion of the recessed portion 182 is provided with a heat insulation layer 185. In the present example, the shaft portion does not have a pulling shaft, and the puncture hole Hh can be expanded only by the self-expansion force of the expansion body 180.

As described above, the medical device 10 according to the present embodiment includes: the expansion body 21 configured to expand and contract in a radial direction; the elongated shaft portion 20 including the distal portion 30 including the proximal end fixing portion 31 to which a proximal end of the expansion body 21 is fixed; and the electrode portion 22 provided along the expansion body 21, in which the expansion body 21 includes the recessed portion 51 recessed radially inward when the expansion body 21 expands and defining the receiving space 51b configured to receive a biological tissue, the recessed portion 51 includes the bottom portion 51a positioned on a radial innermost side, the proximal side upright portion 52 extending radially outward from a proximal end of the bottom portion 51a, and the distal side upright portion 53 extending radially outward from a distal end of the bottom portion 51a, the proximal side upright portion 52 includes the first surface 60 facing the receiving space 51b and the second surface 61 opposite to the first surface 60, the distal side upright portion 53 includes the third surface 62 facing the receiving space 51b and the fourth surface 63 opposite to the third surface 62, one of the proximal side upright portion 52 and the distal side upright portion 53 is an electrode arrangement portion in which the electrode portion 22 is arranged to face the receiving space 51b, and the other of the proximal side upright portion 52 and the distal side upright portion 53 is an opposing surface portion opposing the electrode portion 22, and the expansion body 21 includes the heat insulation layer 71 at least on any one or more of the first surface 60, the second surface 61, the third surface 62, and the fourth surface 63 so as to oppose the electrode portion 22 across the receiving space 51b. In the medical device 10 configured as described above, since the heat insulation layer 71 is provided on the surface opposing the electrode portion 22 across the receiving space 51b, it is possible to make it difficult to propagate, to the blood, heat from the biological tissue raised to a high temperature by the energy imparted from the electrode portion 22 or the heat generation site itself such as the electrode portion 22, and it is possible to reduce the risk of formation of a thrombus.

The expansion body 21 may include the frame 70 defining the shape of the expansion body 21, and the heat insulation layer 71 disposed on the surface of the frame 70, which makes it possible to dispose the heat insulation layer 71 while securing the flexibility of the expansion body 21.

The heat insulation layer 71 may be disposed over the substantially entire surfaces of the inner surface in the expansion direction and the outer surface in the expansion direction of the frame 70, which makes it possible to enhance the heat insulation property of the expansion body 21, and to reliably reduce propagation of the heat associated with cauterization.

The heat insulation layer 107 may be disposed on two or more of the first surface 103, the second surface 104, the third surface 105, and the fourth surface 106 to sandwich the receiving space 102a, which makes it possible to reduce propagation of the heat associated with cauterization on both sides of the recessed portion 102.

The heat insulation layer 71 may be disposed at the bottom portion 51a on an inner surface in the expansion direction or an outer surface in the expansion direction, which makes it possible to reduce heat propagation at the bottom portion 51a of the recessed portion 51.

The expansion body 120 may include a tube 125 covering the frame 123 functioning as the heat insulation layer, which makes it possible to rather easily form the heat insulation layer simply by attaching the tube 125 to the frame 123.

The expansion body 90 may include a frame 91 defining a shape of the expansion body 90, and the frame 91 may include a heat insulation member 92 including the heat insulation layer at least in a region of the recessed portion 93, which makes it possible to rather easily form the heat insulation layer by fixing the heat insulation member 92 to the frame 91.

The medical device 10 according to the present embodiment includes: the expansion body 130 configured to expand and contract in a radial direction; the elongated shaft portion 20 including the distal portion 30 including the proximal end fixing portion 31 to which a proximal end of the expansion body 130 is fixed; the electrode portion 136 provided along the expansion body 130; and the heat insulation cover portion 135 covering at least a part of the expansion body 130, in which the expansion body 130 includes the recessed portion 132 recessed radially inward when the expansion body 130 expands and defining the receiving space 132a configured to receive a biological tissue, the recessed portion 132 includes a bottom portion positioned on a radial innermost side, the proximal side upright portion 132b extending radially outward from a proximal end of the bottom portion, and the distal side upright portion 132c extending radially outward from a distal end of the bottom portion, the electrode portion 136 is arranged in the recessed portion 132 to face the receiving space 132a, and the heat insulation cover portion 135 is configured to cover at least a part of the recessed portion 132 on a surface opposite to a surface facing the receiving space 132a in the vicinity of the electrode portion 136. In the medical device 10 configured in this manner, since a part of the recessed portion 132 on a surface opposite to a surface facing the receiving space 132a is covered with the heat insulation cover portion 135 at least in the vicinity of the electrode portion 136, it is possible to make it difficult to propagate, to the blood, heat from the biological tissue raised to a high temperature by the energy imparted from the electrode portion 136, and it is possible to reduce the risk of formation of a thrombus.

One of the proximal side upright portion 132b and the distal side upright portion 132c is an electrode arrangement portion in which the electrode portion 136 is arranged to face the receiving space 132a, and the other of the proximal side upright portion 132b and the distal side upright portion 132c is an opposing surface portion opposing the electrode portion 136, and the heat insulation cover portion 135 may be disposed on the opposing surface portion on a surface opposite to a surface facing the receiving space 132a, which makes it possible to help prevent the blood from coming into contact with the opposing surface portion, and therefore it is possible to reliably reduce propagation of the heat generated with cauterization.

The expansion body 140 may include a frame 141 defining a shape of the expansion body 140, the medical device 15 may further include a second expansion body 145 configured to expand and contract in a radial direction, including the heat insulation cover portion 147 inside an expansion direction of the frame 141, and the heat insulation cover portion 147 may cover at least a surface of the recessed portion 142 of the frame 141 opposite to a surface facing the receiving space 142a. Due to this, the second expansion body 145 also expands along with the expansion of the expansion body 140, and the recessed portion 142 can be covered with the heat insulation cover portion 147 on a surface opposite to the side facing the receiving space 142a.

The second expansion body 145 may include a second frame 146 defining a shape of the second expansion body 145, and the heat insulation cover portion 147 arranged on at least a part of the second frame 146, which makes it possible to dispose the heat insulation cover portion 147 while securing the flexibility of the second expansion body 145.

The second expansion body 155 may include a mesh in which a large number of wires are knitted, and the heat insulation cover portion 156 arranged on at least a part of the mesh. Since the mesh is configured to flexibly deform in accordance with the shape of the expansion body 150, it is possible to enhance the heat insulation property by bringing the heat insulation cover portion 156 into closer contact to the expansion body 150.

The second expansion body 165 may include the balloon 166 configured to expand in a radial direction, functioning as the heat insulation cover portion, which allows the inside of the expansion body 160 to be covered with the balloon 166, and therefore it is possible to more reliably help prevent the surface of the recessed portion 162 of the frame 161 opposite to the surface facing the receiving space 162a from coming into contact with the blood, and it is also possible to more reliably reduce the propagation of heat.

In the shunt forming method according to the present embodiment, when a voltage is applied to the electrode portion 22, since the recessed portion 51 of the expansion body 21 is insulated by the heat insulation layer 71 or the heat insulation cover portion 135, it is possible to make it difficult to propagate, to the blood, heat associated with cauterization, and it is possible to reduce the risk of formation of a thrombus.

The present disclosure is not limited to the above-described embodiments, and various modifications can be made by those skilled in the art within the technical idea of the present disclosure. In the examples of FIGS. 28 and 29, the expansion bodies 170 and 180 may include a heat insulation cover portion instead of the heat insulation layers 176 and 185.

The detailed description above describes embodiments of a medical device and a shunt forming method that impart energy to a biological tissue. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A medical device comprising:
an expansion body configured to expand and contract in a radial direction;
an elongated shaft portion including a distal portion, the distal portion including a proximal end fixing portion to which a proximal end of the expansion body is fixed;
an electrode portion provided along the expansion body, the expansion body including a recessed portion recessed radially inward when the expansion body expands and defining a receiving space configured to receive a biological tissue;
the recessed portion includes a bottom portion positioned on a radial innermost side, a proximal side upright portion extending radially outward from a proximal end of the bottom portion, and a distal side upright portion extending radially outward from a distal end of the bottom portion;
the proximal side upright portion includes a first surface facing the receiving space and a second surface opposite to the first surface;
the distal side upright portion includes a third surface facing the receiving space and a fourth surface opposite to the third surface;
one of the proximal side upright portion and the distal side upright portion is an electrode arrangement portion in which the electrode portion is arranged to face the receiving space, and an other of the proximal side upright portion and the distal side upright portion is an opposing surface portion opposing the electrode portion; and
the expansion body includes a heat insulation layer at least on one or more of the first surface, the second surface, the third surface, and the fourth surface so as to oppose the electrode portion across the receiving space.

2. The medical device according to claim 1, wherein the expansion body includes a frame defining a shape of the expansion body, and the heat insulation layer is disposed on a surface of the frame.

3. The medical device according to claim 2, wherein the heat insulation layer is disposed over substantially entire surfaces of an inner surface of the frame in an expansion direction and an outer surface of the frame in the expansion direction.

4. The medical device according to claim 2, wherein the heat insulation layer is disposed on two or more of the first surface, the second surface, the third surface, and the fourth surface to sandwich the receiving space.

5. The medical device according to claim 2, wherein the heat insulation layer is disposed at the bottom portion on an inner surface of the frame in an expansion direction or an outer surface of the frame in the expansion direction.

6. The medical device according to claim 2, wherein the expansion body includes a tube covering the frame, and wherein the tube functions as the heat insulation layer.

7. The medical device according to claim 1, wherein
the expansion body includes a frame defining a shape of the expansion body; and
the frame includes a heat insulation member including the heat insulation layer at least in a region of the recessed portion.

8. A medical device comprising:
an expansion body configured to expand and contract in a radial direction;
an elongated shaft portion including a distal portion, the distal portion including a proximal end fixing portion to which a proximal end of the expansion body is fixed;
an electrode portion provided along the expansion body;
a heat insulation cover portion covering at least a part of the expansion body;
the expansion body including a recessed portion recessed radially inward when the expansion body expands and defining a receiving space configured to receive a biological tissue;
the recessed portion including a bottom portion positioned on a radial innermost side, a proximal side upright portion extending radially outward from a proximal end of the bottom portion, and a distal side upright portion extending radially outward from a distal end of the bottom portion;
the electrode portion is arranged in the recessed portion to face the receiving space; and
the heat insulation cover portion is configured to cover at least a part of the recessed portion on a surface opposite to a surface facing the receiving space in a vicinity of the electrode portion.

9. The medical device according to claim 8, wherein
one of the proximal side upright portion and the distal side upright portion is an electrode arrangement portion in which the electrode portion is arranged to face the receiving space, and an other of the proximal side upright portion and the distal side upright portion is an opposing surface portion opposing the electrode portion; and
the heat insulation cover portion is disposed on the opposing surface portion on a surface opposite to a surface facing the receiving space.

10. The medical device according to claim 8, wherein
the expansion body includes a frame defining a shape of the expansion body;
the medical device further includes a second expansion body configured to expand and contract in a radial direction, including the heat insulation cover portion inside an expansion direction of the frame; and
the heat insulation cover portion covers at least a surface of the recessed portion of the frame opposite to a surface facing the receiving space.

11. The medical device according to claim 10, wherein the second expansion body includes a second frame defining a shape of the second expansion body, and the heat insulation cover portion arranged on at least a part of the second frame.

12. The medical device according to claim 8, wherein the second expansion body includes a mesh in which a large number of wires are knitted, and the heat insulation cover portion arranged on at least a part of the mesh.

13. The medical device according to claim 8, wherein the second expansion body includes a balloon configured to expand in a radial direction, functioning as the heat insulation cover portion.

14. A method of forming a shunt in an atrial septum using a medical device including an expansion body configured to expand and contract in a radial direction, an elongated shaft portion including a distal portion, the distal portion including a proximal end fixing portion to which a proximal end of the expansion body is fixed, and an electrode portion provided along the expansion body, the method comprising:
expanding the expansion body to include a recessed portion recessed radially inward and defining a receiving space configured to receive a biological tissue;
arranging the recessed portion in a puncture hole formed in an atrial septum to receive the biological tissue surrounding the puncture hole in the receiving space defined by the recessed portion, and to bring the electrode portion into contact with the biological tissue, the electrode portion being arranged in the recessed portion to face the receiving space; and
cauterizing the biological tissue by applying a voltage to the electrode portion in a state in which at least a part of the recessed portion includes a heat insulation layer or in a state in which at least a part of the recessed portion is covered with a heat insulation cover portion in a vicinity of the electrode portion.

15. The method according to claim 14, further comprising:
confirming hemodynamics in a vicinity of the puncture hole before the cauterization of the biological tissue

16. The method according to claim 15, further comprising:
confirming hemodynamics in the vicinity of the puncture hole after the cauterization of the biological tissue

17. The method according to claim 14, further comprising:
decreasing a diameter of the expansion body;
storing the expansion body in a storage sheath; and
removing the expansion body stored in the storage sheath from the puncture hole.

18. The method according to claim 14, further comprising:
suppressing a propagation of heat generated by the cauterization of the biological tissue with a heat insulation layer on a surface of a frame of the expansion body.

19. The method according to claim 18, further comprising:
suppressing the propagation of the heat generated by the cauterization of the biological tissue with the heat insulation layer over substantially entire surfaces of an inner surface of the frame in an expansion direction and an outer surface of the frame in the expansion direction.

20. The method according to claim 18, further comprising:
suppressing the propagation of the heat generated by the cauterization of the biological tissue with the heat insulation layer on two or more of a first surface, a second surface, a third surface, and a fourth surface, the recessed portion includes a bottom portion positioned on a radial innermost side, a proximal side upright portion extending radially outward from a proximal end of the bottom portion, and a distal side upright portion extending radially outward from a distal end of the bottom portion, the proximal side upright portion includes the first surface facing the receiving space and the second surface opposite to the first surface, and the distal side upright portion includes the third surface facing the receiving space and the fourth surface opposite to the third surface to sandwich the receiving space.
